(19) United States
(12) Patent Application Publication
(10) Pub. No.: US 2023/0167421 A1
(43) Pub. Date: Jun. 1, 2023

(54) Variants of a DNA Polymerase of the Polx Family

(71) Applicant: DNA Script, Le Kremlin-Bicêtre (FR)

(72) Inventors: Thomas Ybert, Paris (FR); Marc Delarue, Versailles (FR)

(21) Appl. No.: 17/815,490

(22) Filed: Jul. 27, 2022

Related U.S. Application Data
(63) Continuation of application No. 16/309,414, filed on Dec. 12, 2018, now abandoned.

(30) Foreign Application Priority Data
Jun. 14, 2016 (FR) 165547

(51) Int. Cl. C12N 9/12; C12N 15/70; C12P 19/34

(52) U.S. Cl. CPC C12N 9/1264; C12N 15/70; C12P 19/34; C12Y 207/07031

(57) ABSTRACT

The invention relates to variants of a DNA polymerase of the polX family capable of synthesizing a nucleic acid molecule without a template strand, or of a functional fragment of such a polymerase, comprising at least one mutation of a residue in at least one specific position, and to uses of said variants, in particular for the synthesis of nucleic acid molecules comprising 3′-OH modified nucleotides.

### CROSS-REFERENCE TO RELATED APPLICATIONS

This application is a continuation of U.S. Pat. Application Serial No. 16/309,414, filed Dec. 12, 2018, which is a 371 national phase of International Application Serial No. PCT/FR2017/051519, filed Jun. 13, 2017, which claims priority to French Patent Application Serial No. 1655475, filed Jun. 14, 2016, which applications are incorporated herein by reference in their entirety.

### INCORPORATION BY REFERENCE OF SEQUENCE LISTING

A Sequence Listing is provided herewith as a Sequence Listing XML, DNAS-001CON_SEQ_LIST_CORRECTED created on Sep. 30, 2022 and having a size of 26,958 bytes. The contents of the Sequence Listing XML are incorporated herein by reference in their entirety.

### INTRODUCTION

The present invention relates to the field of enzyme improvement. The present invention relates to an improved variant of a DNA polymerase of the polX family, to a nucleic acid coding for this variant, to the production of this variant in a host cell, to the use thereof for the synthesis of a nucleic acid molecule without a template strand, and to a kit for the synthesis of a nucleic acid molecule without a template strand.

The chemical synthesis of nucleic acid fragments is a widely used laboratory technique (Adams et al., 1983, J. Amer. Chem. Soc. 105:661; Froehler et al., 1983; Tetrahedron Lett. 24:3171). It makes it possible to rapidly obtain nucleic acid molecules comprising the desired nucleotide sequence. In contrast to enzymes which carry out the synthesis in the 5′ to 3′ direction, the chemical synthesis is carried out in the 3′ to 5′ direction. However, the chemical synthesis has certain limits. In fact, it requires the use of multiple solvents and reagents. In addition, it only makes it possible to obtain short nucleic acid fragments which then have to be assembled to one another to obtain the desired final nucleic acid strands.

An alternative solution using enzymes for carrying out the coupling reaction between nucleotides from an initial nucleic acid fragment (primer) and in the absence of a template strand has been developed. Several polymerase enzymes appear to be suitable for this type of synthesis methods.

A very large number of DNA polymerases exists, which are capable of catalyzing the synthesis of a nucleic acid strand in the presence or absence of a template strand. Thus, the DNA polymerases of the polX family are involved in a large range of biological processes, in particular in DNA repair mechanisms or mechanisms for the correction of errors appearing in DNA sequences. These enzymes are capable of inserting nucleotides, which have undergone excisions after the identification of sequence errors, in the nucleic acid strands. The DNA polymerases of the polX family comprise the DNA polymerases β (Pol β λ (Pol λ), µ (Pol µ), yeast IV (Pol IV), and the terminal deoxyribonucleotidyl transferase (TdT). TdT in particular is used very widely in the methods of enzymatic synthesis of nucleic acid molecules.

However, usually these DNA polymerases allow only the incorporation of natural nucleotides. In all cases, the natural DNA polymerases lose their catalytic activity in the presence of non-natural nucleotides and in particular 3′-OH modified nucleotides which exhibit greater steric hindrance than the natural nucleotides.

However, the use of modified nucleotides can turn out to be useful for certain specific applications. Therefore, enzymes that are capable of catalyzing the synthesis of a nucleic acid strand by incorporating such nucleotides had to be developed. Thus, DNA polymerase variants that can function with nucleotides comprising considerable structural modifications have been developed.

However, the currently available variants are not entirely satisfactory, in particular since they exhibit low activity and since they are only compatible with enzymatic synthesis on the laboratory scale. Thus, a need exists for DNA polymerases capable of synthesizing, if possible on an industrial scale, a nucleic acid in the absence of a template strand and using modified nucleotides.

### SUMMARY OF THE INVENTION

The present invention overcomes certain technological barriers which prevent the use on an industrial scale of DNA polymerases for the enzymatic synthesis of nucleic acids.

The present invention thus proposes DNA polymerases of the polX family capable of synthesizing a nucleic acid in the absence of a template strand and suitable for using modified nucleotides. The variants developed exhibit capabilities of incorporation of modified nucleotides which are much greater than those of the natural DNA polymerases from which they are derived. In particular, the DNA polymerase variants which are the subject matter of the present invention are particularly effective for the incorporation of nucleotides having modifications of the sugar. In fact, the inventors have developed variants having an increased catalytic pocket volume in comparison to that of the DNA polymerases from which they are derived, promoting the incorporation of modified nucleotides exhibiting greater steric hindrance than the natural nucleotides. More particularly, the DNA polymerase variants of the polX family which are the subject matter of the present invention comprise at least one mutation on an amino acid intervening directly at the level of the catalytic cavity of the enzyme, or enabling the deformation of the contours of this cavity in order to accommodate the steric hindrance due to the modifications present at the level of the nucleotides. For example, the mutations introduced enable the enlargement of the catalytic cavity of the enzyme in which the 3′-OH end of the modified nucleotides is accommodated. Alternatively or additionally, the mutations carried out enable the inflation or increase of the volume of the catalytic activity, the increase in the access to the catalytic pocket by the 3′-OH modified nucleotides and/or they confer the necessary flexibility to the structure of the enzyme to enable it to accommodate modifications resulting in great steric hindrance of the 3′-OH modified nucleotides. As a result of such mutations, once the polymerase is bound to the nucleic acid fragment to be elongated, the modified nucleotide penetrates into the core of the catalytic pocket whose access is widened and it takes on an optimal spatial conformation in said catalytic pocket, a phosphodiester bond forming between the 3′-OH end of the last nucleotide of the nucleic acid strand and the 5′-triphosphate end of the modified nucleotide.

Thus, the subject matter of the invention is a variant of a DNA polymerase of the polX family capable of synthesizing a nucleic acid molecule without a template strand, or a variant of a functional fragment of such a polymerase, said variant comprising at least one mutation of a residue in at least one position selected from the group consisting of T331, G332, G333, F334, R336, K338, H342, D343, V344, D345, F346, A397, D399, D434, V436, A446, L447, L448, G449, W450, G452, R454, Q455, F456, E457, R458, R461, N474, E491, D501, Y502, I503, P505, R508, N509 and A510, or a functionally equivalent residue, the positions indicated being determined by alignment with SEQ ID No. 1.

In a particular embodiment, the variant is capable of synthesizing a DNA strand or an RNA strand.

The present invention relates in particular to a variant of a DNA polymerase of the polX family and in particular of a Pol IV from yeast, Pol µ or wild-type TdT, and comprising the selected mutation(s). In a particular embodiment, the variant according to the present invention is a variant of the TdT of sequence SEQ ID No. 1 or a homologous sequence which has at least 70%, 80%, 85%, 90%, 95%, 96%, 97%, 98%, 99% identity with the sequence of the SEQ ID No. 1, and it carries the selected mutation(s).

The invention also relates to a nucleic acid coding for a variant of a DNA polymerase of the polX family according to the present invention, to an expression cassette comprising a nucleic acid according to the present invention, and to a vector comprising a nucleic acid or an expression cassette according to the present invention. The nucleic acid coding for the variant of the present invention can be the nucleic acid of mature form or of the precursor form of the DNA polymerase according to the invention.

The present invention also relates to the use of a nucleic acid, of an expression cassette or of a vector according to the present invention for transforming or transfecting a host cell. It further relates to a host cell comprising a nucleic acid, an expression cassette or a vector coding for a DNA polymerase of the polX family according to the present invention. It relates to the use of such a nucleic acid, of such an expression cassette, of such a vector or of such a host cell for producing a variant of a DNA polymerase of the polX family according to the present invention.

It also relates to a method for producing a variant of the DNA polymerase of the polX family according to the present invention, comprising the transformation or the transfection of a host cell by a nucleic acid, an expression cassette or a vector according to the present invention, the culturing of the transformed/transfected host cell under culture conditions enabling the expression of the nucleic acid coding for said variant, and optionally, the harvesting of a variant of a DNA polymerase of the polX family produced by the host cell.

The host cell can be prokaryotic or eukaryotic. In particular, the host cell can be a microorganism, preferably a bacterium, a yeast or a mushroom. In an embodiment, the host cell is a bacterium, preferably E. coli. In another embodiment, the host cell is a yeast, preferably P. pastoris or K. lactis. In another embodiment, the host cell is a mammalian cell, preferably a COS7 or CHO cell.

The invention also relates to the use of a variant of a DNA polymerase of the polX family according to the present invention for synthesizing a nucleic acid molecule without a template strand, from 3′-OH modified nucleotides. Naturally, the variant of a DNA polymerase of the polX family according to the present invention can also be used, in the context of the invention, for synthesizing a nucleic acid molecule without a template strand, from non modified nucleotides or from a mixture of modified and non modified nucleotides.

The invention also proposes a method for the enzymatic synthesis of a nucleic acid molecule without a template strand, according to which a primer strand is brought in contact with at least one nucleotide, preferably a 3′-OH modified nucleotide, in the presence of a variant of a DNA polymerase of the polX family according to the invention. The carrying out of the method can take place in particular by using a purified variant, a culture medium of a host cell which has been transformed to express said variant, and/or a cell extract of such a host cell.

The invention also relates to a kit for the enzymatic synthesis of a nucleic acid molecule without a template strand, comprising at least one variant of a DNA polymerase of the polX family according to the invention, nucleotides, preferably 3′-OH modified nucleotides, and optionally at least one primer strand, or nucleotide primer, and/or a reaction buffer.

### DESCRIPTION OF THE FIGURES

FIG. 1: SDS-PAGE gel of fractions of a TdT variant according to an embodiment example of the invention (M: Molecular weight marker; 1: Centrifugate before loading; 2: Centrifugate after loading; 3: Washing buffer after loading; 4: Elution fraction 3 mL; 5: Elution fraction 30 mL; 6: Elution peak compilation; 7: Concentration);

FIGS. 2A-2D: Alignment of the amino acid sequences of the Homo sapiens DNA polymerases Pol µ (UniProtKB Q9NP87), Pan troglodytes Pol µ (UniProtKB H2QUI0), Mus musculus Pol µ (UniProtKB Q924W4), Canis lupus familiaris Pol µ (UniProtKB F1P657), Mus musculus TdT (UniProtKB Q3UZ80), Gallus gallus TdT (UniProtKB P36195) and Homo sapiens TdT (UniProtKB P04053) obtained by means of the online alignment software (http://multalin.toulouse.infra.fr/multalin/multalin.html);

FIG. 3: Comparison of the activity of a truncated wild-type TdT of sequence SEQ ID No. 3 and of several variants of this truncated TdT comprising different substitutions given in table 1, in the presence of a primer which has been radioactively labeled beforehand at the 5′ end and of 3′-O-amino-2′,3′-dideoxyadenosine-5′-triphosphate modified nucleotides (ONH2 gel) or 3′-biot-EDA-2′,3′-dideoxyadenosine-5′-triphosphate modified nucleotides (Biot-EDA gel); on SDS-PAGE gel (No: no enzyme present; wt: truncated wild-type TdT of sequence SEQ ID No. 3; DSi: Variants i defined in table 1);

FIG. 4: Study of the activity of the variant DS 124 according to the invention (see table 1), in the presence of a primer which has been radioactively labeled beforehand at the 5′ end and different 3′-O-amino-2′,3′-dideoxyadenosine-5′-triphosphate modified nucleotides on SDS-PAGE gel;

FIG. 5: Study of the activity of the variants DS22, DS24, DS124, DS125, DS126, DS 127 and DS 128 in the presence of a primer which has been radioactively labeled beforehand at the 5′ end and different 3′-O-amino-2′,3′-dideoxyadenosine-5′-triphosphate modified nucleotides on SDS-PAGE gel;

FIG. 6: Synthesis of a DNA strand of sequence: 5′-GTACGCTAGT-3′ (SEQ ID No. 15) after the primer of sequence 5′-AAAAAAAAAAGGGG-3′ (SEQ ID No. 14) by means of a variant of the TDT according to the invention having the combination of substitutions R336N -R454A - E457G (DS125).

### DETAILED DESCRIPTION OF THE INVENTION

### Definitions

The amino acids are represented in this document by a one-letter or three-letter code according to the following nomenclature: A: Ala (alanine); R: Arg (arginine); N: Asn (asparagine); D: Asp (aspartic acid); C: Cys (cysteine); Q: Gln (glutamine); E: Glu (glutamic acid); G: Gly (glycine); H: His (histidine); I: Ile (isoleucine); L: Leu (leucine); K: Lys (lysine); M: Met (methionine); F: Phe (phenylalanine); P: Pro (proline); S: Ser (serine); T: Thr (threonine); W: Trp (tryptophan); Y: Tyr (tyrosine); V: Val (valine).

“Percentage of identity” between two nucleic acid or amino acid sequences in the sense of the present invention is understood to designate a percentage of nucleotides or of amino acid residues which are identical between the two sequences to be compared, which is obtained after the best alignment, this percentage being purely statistical and the differences between the two sequences being distributed randomly and over their entire length. The best alignment or optimal alignment is the alignment for which the percentage of identity between the two sequences to be compared, as calculated below, is the highest. The comparisons of sequences between two nucleic acid or amino acid sequences are traditionally carried out by comparing these sequences after having aligned them in an optimal manner, said comparison being carried out by segment or by comparison window in order to identify and compare the local regions of sequence similarity. The optimal alignment of the sequences for the comparison can be carried out, besides manually, by means of the local homology algorithm of Smith and Waterman (1981) (Ad. App. Math. 2:482), by means of the local homology algorithm of Neddleman and Wunsch (1970) (J. Mol. Biol. 48:443), by means of the similarity search method of Pearson and Lipman (1988) (Proc. Natl. Acad. Sci. USA 85:2444), by means of computer software using these algorithms (GAP, BESTFIT, FASTA and TFASTA in the Wisconsin Genetics Software Package, Genetics Computer Group, 575 Science Dr., Madison, WI), by means of the online alignment software Mutalin (http://multalin.toulouse.inra.fr/multalin/multalin.html; 1988, Nucl. Acids Res., 16 (22), 10881-10890). The percentage of identity between two nucleic acid or amino acid sequences is determined by comparing these two sequences which are aligned in an optimal manner by comparison window in which the region of the nucleic acid or amino acid sequence to be compared can comprise additions or deletions with respect to the reference sequence for an optimal alignment between these two sequences. The percentage of identity is calculated by determining the number of identical positions for which the nucleotide or the amino acid residue is identical between the two sequences, by dividing this number of identical positions by the total number of positions in the comparison window and by multiplying the result obtained by 100 in order to obtain the percentage of identity between these two sequences.

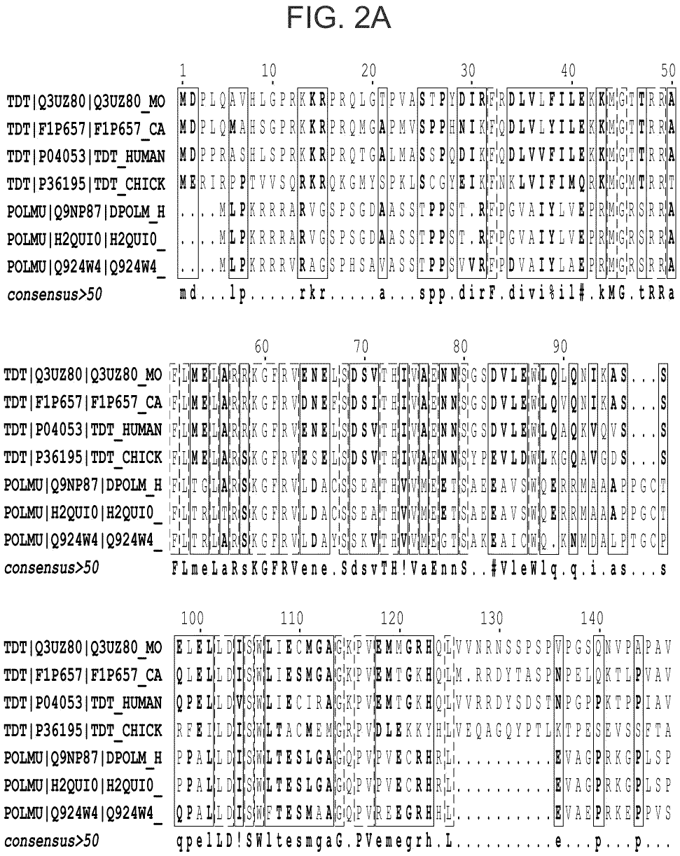

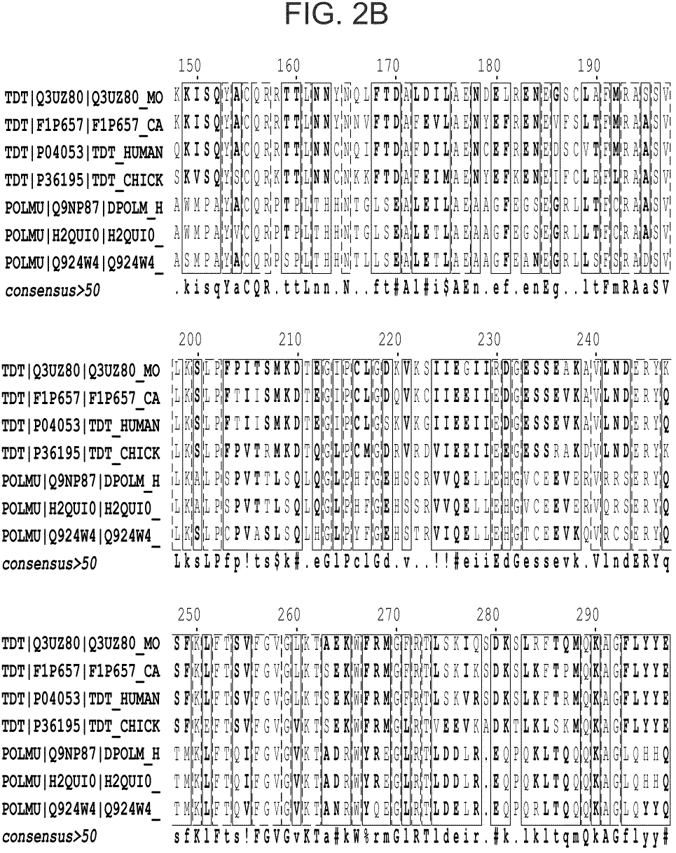

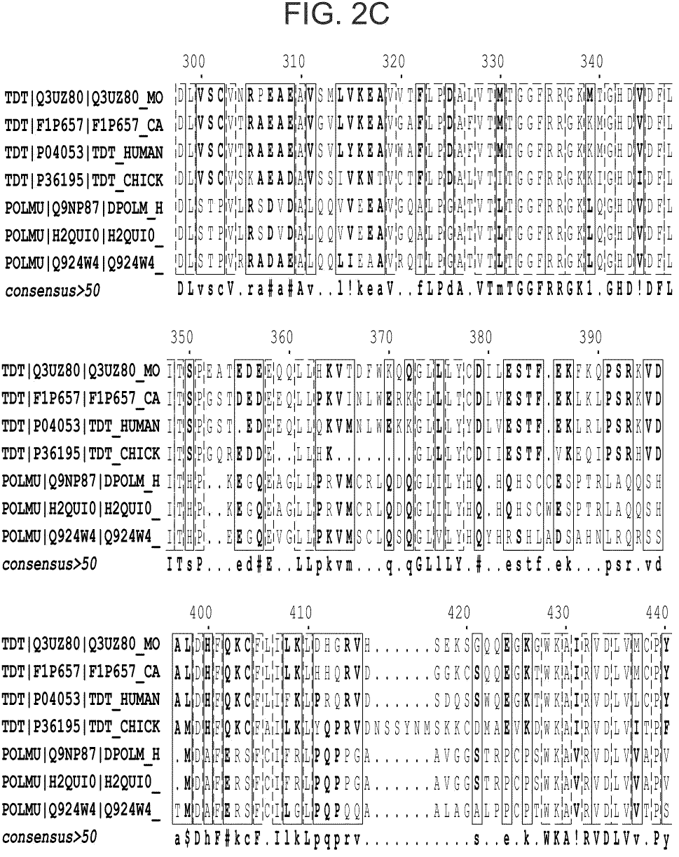

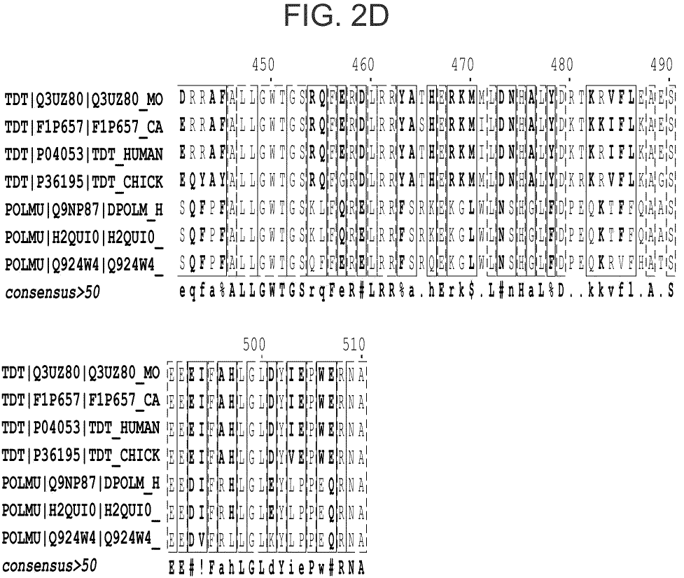

The variants which are the subject matters of the present invention are described as a function of their mutations on specific residues, the positions of which are determined by alignment with, or reference to, the enzymatic sequence SEQ ID No. 1. In the context of the invention, any variant carrying these same mutations on functionally equivalent residues is also covered. “Functionally equivalent residue” is understood to mean a residue in a sequence of a DNA polymerase of the polX family having a sequence homologous to SEQ ID No. 1 and having an identical functional role. The functionally equivalent residues are identified using sequence alignments which are carried out, for example, by means of the online alignment software Mutalin (http://multalin.toulouse.inra.fr/multalin/multalin.html; 1988, Nucl. Acids Res., 16 (22), 10881-10890). After alignment, the functionally equivalent residues are in homologous positions on the different sequences considered. The alignments of sequences and the identification of functionally equivalent residues can occur between any DNA polymerases of the polX family and their natural variants, including interspecies variants. For example, the residue L40 of human TdT (UniProtKB P04053) is functionally equivalent to the residue M40 of chicken TdT (UniProtKB P36195) and to the residue V40 of Pan troglodytes Polµ (UniProtKB H2QUI0), said residues being considered after alignment of the sequences (FIG. 2).

“Functional fragment” is understood to mean a fragment of a DNA polymerase of the polX family exhibiting the DNA polymerase activity. The fragment can comprise 100, 200, 300, 310, 320, 330, 340, 350, 360, 370, 380 or more consecutive amino acids of a DNA polymerase of the polX family. Preferably, the fragment comprises 380 consecutive amino acids of a DNA polymerase of the polX family consisting of the catalytic fragment of said enzyme.

The terms “mutant” and “variant” can be used interchangeably to refer to polypeptides derived from DNA polymerases of the polX family, or derivatives of functional fragments of such DNA polymerases, and in particular from a TdT such as the murine TdT according to the sequence SEQ ID No. 1, and comprising an alteration, namely a substitution, an insertion and/or a deletion in one or more positions and having a DNA polymerase activity. The variants can be obtained by various techniques well known in the art. In particular, examples of techniques for modifying the DNA sequence coding for the wild-type proteins comprise, without being limited thereto, directed mutagenesis, random mutagenesis, and the construction of synthetic oligonucleotides.

The term “modification” or “mutation” as used here with respect to a position or an amino acid residue means that the amino acid in the position considered has been modified with respect to the amino acid of the reference wild-type protein. Such modifications comprise the substitutions, deletions and/or insertions of one or more amino acids, and in particular 1 to 5, 1 to 4, 1 to 3, 1 to 2 amino acids, in one or more positions, and in particular in 1, 2, 3, 4, 5 or more positions.

The term “substitution,” in relation to a position or an amino acid residue, means that the amino acid in the particular position has been replaced by another amino acid than the wild-type or parent DNA polymerase. Preferably, the term “substitution” denotes the replacement of one amino acid residue by another amino acid residue selected from the 20 standard natural amino acid residues, the rare amino acid residues of natural origin (for example, hydroxyproline, hydroxylysine, allohydroxylysine, 6-N-methyllysine, N-ethylglycine, N-methylglycine, N-ethylasparagine, allo-isoleucine, N-methylisoleucine, N-methylvaline, pyroglutamine, aminobutyric acid, ornithine), and the rare non-natural amino acid residues, often produced synthetically (for example, norleucine, norvaline and cyclohexylalanine). Preferably, the term “substitution” denotes the replacement of one amino acid residue by another amino acid residue selected from the 20 standard amino acid residues of natural origin (G, P, A, V, L, I, M, C, F, Y, W, H, K, R, Q, N, E, D, S and T). The substitution can be a conservative or non-conservative substitution. The conservative substitutions occur within the same group of amino acids, among the basic amino acids (arginine, lysine and histidine), the acidic amino acids (glutamic acid and aspartic acid), the polar amino acids (glutamine and asparagine), the hydrophobic amino acids (methionine, leucine, isoleucine and valine), the aromatic amino acids (phenylalanine, tryptophan and tyrosine), and the small amino acids (glycine, alanine, serine and threonine). In the present document, the following terminology is used to designate a substitution: R454F indicates that the amino acid residue in position 454 of the SEQ ID No. 1 (arginine, R) is replaced by a phenylalanine (F). N474S/T/N/Q means that the amino acid in position 474 (asparagine, N) can be replaced by a serine (S), a threonine (T), an asparagine (N) or a glutamine (Q). The “+” indicates a combination of substitutions.

The invention relates to variants of DNA polymerases of the polX family (EC 2.7.7.7; Advances in Protein Chemistry, Vol. 71, 401-440) which are capable of synthesizing a nucleic acid molecule without a template strand, and in particular a DNA or RNA strand. The DNA polymerases of the polX family comprise in particular the DNA polymerase Polβ (UniProt P06746 in humans; Q8K409 in mice), Polσ, Polλ (UniProt Q9UGP5 in humans; Q9QUG2 and Q9QXE2 in mice) and Polµ (UniProt Q9NP87 in humans; Q9JIW4 in mice), Pol4 (UniProt A7TER5 in the yeast Vanderwaltozyma polyspora; P25615 in the yeast Saccharomyces cerevisiae) and the terminal deoxyribonucleotidyl transferase or TdT (EC 2.7.7.31; UniProt P04053 in humans; P09838 in mice).

The invention relates more particularly to a variant of a DNA polymerase of the polX family capable of synthesizing a nucleic acid molecule without a template strand, or to a variant of a functional fragment of such a polymerase, said variant comprising at least one mutation of a residue in at least one position selected from the group consisting of T331, G332, G333, F334, R336, K338, H342, D343, V344, D345, F346, A397, D399, D434, V436, A446, L447, L448, G449, W450, G452, R454, Q455, F456, E457, R458, R461, N474, E491, D501, Y502, I503, P505, R508, N509 and A510, or a functionally equivalent residue, the positions indicated being determined by alignment with, or reference to, the sequence SEQ ID No. 1.

In an embodiment, the variant is capable of synthesizing a DNA strand and/or an RNA strand.

“Comprise at least one mutation” or “comprising at least one mutation” is understood to mean that the variant has one or more mutations as indicated with respect to the polypeptide sequence SEQ ID No. 1, but it can have other modifications, in particular substitutions, deletions or additions.

In general, the mutation of one or more residues in the above positions makes it possible to enlarge the catalytic pocket (by targeting, for example, the positions W450, D434, D435, H342, D343, T331, R336, D399, R461, and/or R508) and to increase the accessibility to the catalytic pocket (by targeting, for example, the positions R458, E455, R454, A397, K338, and/or N509) and/or it confers greater flexibility to the structure of the enzyme, enabling it to receive modified nucleotides exhibiting large steric hindrance (by targeting, for example, the positions V436, F346, V344, F334, M330, L448, E491, E457 and/or N474).

The variants which are the subject matters of the present invention can be variants of Pol IV, Pol µ, Polβ, Polλ or of TdT, preferably variants of Pol IV, Pol µ, or TdT. Alternatively, the variants can be variants of chimeric enzymes, combining, for example, portions of different sequences of at least two DNA polymerases of the polX family.

In a particular embodiment, the variant has at least 60% identity with the sequence according to SEQ ID No. 1, preferably at least 70%, 80%, 85%, 90%, 95%, 96%, 97%, 98%, 99% and less than 100% identity with the sequence according to SEQ ID No. 1.

According to the invention, the mutation can consist of a substitution, a deletion or an addition of one or more amino acid residues. In the deletion case, the annotation X is used, which indicates that the codon coding for the residue considered is replaced by a STOP codon; all the following amino acids as well as the residue in question are thus deleted. Thus, the mutation D501X means that the enzyme ends at the residue preceding the aspartic acid (D) in position 501, that is to say the leucine (L) in position 500, all the residues beyond having been deleted. The annotation ∅, on the other hand, denotes a single point deletion of the residue considered. Thus, the mutation D501∅ means that the aspartic acid (D) in position 501 has been deleted.

Preferably, the variant according to the invention comprises at least one mutation of a residue in at least one position selected from the group consisting of T331, G332, G333, F334, R336, D343, L447, L448, G449, W450, G452, R454, Q455, E457 and R508, or a functionally equivalent residue, preferably at least one mutation of a residue in at least one position selected from the group consisting of R336, R454, E457, or a functionally equivalent residue, the positions indicated being determined by alignment with SEQ ID No. 1.

In a particular embodiment, said variant comprises at least one mutation of a residue in at least two positions selected from the group consisting of R336, R454 and E457, preferably a mutation of a residue in said three positions R336, R454 and E457, or a functionally equivalent residue, the positions indicated being determined by alignment with SEQ ID No. 1.

In a particular embodiment, the variant moreover comprises at least one mutation of a residue in at least the semi-conserved region of sequence X₁X₂GGFR₁R₂GKX₃X₄ (SEQ ID No. 4), in which
X₁ represents a residue selected from M, I, V, L
X₂ represents a residue selected from T, A, M, Q
X₃ represents a residue selected from M, K, E, Q, L, S, P, R, D
X₄ represents a residue selected from T, I, M, F, K, V, Y, E, Q, H, S, R, D.

Preferably, said variant has at least one substitution of a residue in at least one position R₁, R₂ and/or K of the semi-conserved region of sequence SEQ ID No. 4.

In another particular embodiment, the variant moreover comprises at least one mutation of a residue in at least one semi-conserved region of sequence X₁X₂LGX₃X₄GSR₁X₅X₆ER₂ (SEQ ID No. 5) in which
X₁ represents a residue selected from A, C, G, S
X₂ represents a residue selected from L, T, R
X₃ represents a residue selected from W, Y
X₄ represents a residue selected from T, S, I
X₅ represents a residue selected from Q, L, H, F, Y, N, E, D or ∅
X₆ represents a residue selected from F, Y

Preferably, said variant has at least one substitution of a residue in at least one position S, R₁ and/or E of the semi-conserved region of sequence SEQ ID No. 5.

In another particular embodiment, the variant moreover comprises at least one mutation of a residue in at least one semi-conserved region of sequence LX₁YX₂X₃PX₄X₅RNA (SEQ ID No. 6) in which
X₁ represents a residue selected from D, E, S, P, A, K
X₂ represents a residue selected from I, L, M, V, A, T
X₃ represents a residue selected from E, Q, P, Y, L, K, G, N
X₄ represents a residue selected from W, S, V, E, R, Q, T, C, K, H
X₅ represents a residue selected from E, Q, D, H, L.

Preferably, said variant has at least one deletion of the residue in position X₁ and/or at least one substitution in positions R and/or N of the semi-conserved region of sequence SEQ ID No. 6.

In a particular embodiment, the variant comprises a substitution of a residue in at least one position selected from the group consisting of R336, K338, H342, A397, S453, R454, E457, N474, D501, Y502, I503,R508 and N509, or a functionally equivalent residue, preferably a substitution of a residue in at least one position selected from the group consisting of R336, A397, R454, E457, N474, D501, Y502 and I503,or a functionally equivalent residue, more preferably at least one substitution of a residue in at least one position selected from the group consisting of R336, R454 and E457, or a functionally equivalent residue, the positions indicated being determined by alignment with SEQ ID No. 1.

The invention preferably relates to a variant of a DNA polymerase of the polX family comprising at least one substitution from the group consisting of R336K/H/G/N/D, K338A/C/G/S/T/N, H342A/C/G/S/T/N, A397R/H/K/D/E, S453A/C/G/S/T, R454F/Y/W/A, E457G/N/S/T, N474S/T/N/Q, D501A/G/X, Y502A/G/X, I503A/G/X, R508A/C/G/S/T, N509A/C/G/S/T. In a particular embodiment, the variant comprises a substitution of a residue in at least two positions selected from the group consisting of R336, R454, E457, or a functionally equivalent residue, preferably a substitution of a residue in said three positions, or a functionally equivalent residue, the positions indicated being determined by alignment with SEQ ID No. 1. In particular, the substitutions are selected from the group consisting of R336K/H/G/N/D, R454F/Y/W/A and E457N/D/G/S/T, preferably from the group consisting of R336N/G, R454A and E457G/N/S/T.

In an embodiment, the variant comprises at least one substitution according to E457G/N/S/T.

Advantageously, the variant comprises a combination of substitutions selected from the group mentioned above. The combination can consist of 2, 3, 4, 5, 6, 7, 8, 9, 10 or 11 substitutions selected from this group.

The invention relates more particularly to variants of a DNA polymerase of the polX family which are capable of synthesizing a nucleic acid molecule, such as a DNA or RNA strand without a template strand, or of a functional fragment of such a polymerase, said variants comprising at least one combination of mutations described in table 1, the positions indicated being determined by alignment with SEQ ID No. 1.

In an embodiment, the variant of a DNA polymerase of the polX family comprises a combination of substitutions from R336G - E457N; R336N - E457N; R336N - R454A - E457N; R336N - E454A - E457G; R336N - E457G; and R336G - R454A - E457N.

TABLE 1

Examples of combinations of mutations of variants of a DNA polymerase of the polX family

| | |
|---|---|
| | Combinations of mutations |
| DS1 | R454F - E457N - A397D |
| DS2 | R454F - E457N |
| DS3 | R454Y - E457N - A397D |
| DS4 | R454Y - E3457N |
| DS5 | R454W - E457N - A397D |
| DS6 | R454W - E457N |
| DS7 | R335A - E457N - A397D |
| DS8 | R335A - E457N |
| DS9 | R335G - E457N - A397D |
| DS10 | R335G - E457N |
| DS11 | R335N - E457N - A397D |
| DS12 | R335N - E4575N |
| DS13 | R335D - E4S7N - A397D |
| DS14 | R335D - E457N |
| DS15 | R336K - E457N - A397D |
| DS16 | R336K - E457N |
| DS17 | R336H - E457N - A397D |
| DS18 | R336H - E457N |
| DS21 | R335G - E457N - A397D |
| DS22 | R336G - E457N |
| DS23 | R336N - E457N - A397D |
| DS24 | R336N - E4S7N |
| DS25 | R336D - E457N - A397D |
| DS26 | R336D - E457N |
| DS27 | R454A - E457N |
| DS28 | R454A - E457A |
| DS29 | R454A - E457G |
| DS30 | R454A - E457D |
| DS31 | E457N |
| DS32 | E4570 |
| DS33 | R454A - E457N - A397D |
| DS34 | R454A - E457N - A3975 |
| DS35 | R454A - E457N - N474S |
| DS36 | R454A - E457D - A397D |
| DS37 | D501X |
| DS38 | D501X - E457N |
| DS39 | D501X - E457N - A397D |
| DS40 | R454F - E4575 - A397D |
| DS41 | R454F - E457S |
| DS42 | R454Y - E4575 - A377D |
| DS43 | R454Y - E457S |
| DS44 | R454W - E4575 - A397D |
| DS45 | R454W - E457S |
| DS46 | R335A - E457S - A397D |
| DS47 | R335A - E457S |
| DS48 | R335G - E457S - A397D |
| DS49 | R335G - E457S |
| DS50 | R335N - E457S - A397D |
| DS51 | R335N - E457S |
| DS52 | R335D - E457S - A397D |
| DS53 | R335D - E457S |
| DS54 | R336K - E457S - A397D |
| DS55 | R336K - E457S |
| DS56 | R336H - E457S - A397D |
| DS57 | R336H - E457S |
| DS60 | R336G - E457S - A397D |
| DS61 | R336G - E457S |
| DS62 | R336N - E4575 - A397D |
| DS63 | R336N - E4575 |
| DS64 | R336D - E457S - A397D |
| DS65 | R336D - E457S |
| DS66 | R454A - E457S |
| DS70 | E457S |
| DS72 | R454A - E457S - A397D |
| DS73 | R454A - E457S - A397K |
| DS74 | R454A - E457S-N474S |
| DS75 | D501X - E457S |
| DS76 | D501X - E457S - A397D |
| DS77 | R454F - E457T - A397D |
| DS78 | R454F - E457T |
| DS79 | R454Y - E457T - A397D |
| DS80 | R454Y - E457T |
| DS81 | R454W - E457T - A397D |
| DS82 | R454W - E457T |
| DS83 | R335A - E457T - A397D |
| DS84 | R335A - E457T |
| DS85 | R335G - E457T - A397D |
| DS86 | R335G - E457T |
| DS87 | R335N - E457T - A397D |
| DS88 | R335N - E457T |
| DS89 | R335D - E457T - A397D |
| DS90 | R335D - E457T |
| DS91 | R336K - E457T - A397D |
| DS92 | R336K - E457T |
| DS93 | R336H - E457T - A397D |
| DS94 | R336H - E457T |
| DS97 | R336G - E457T - A397D |
| DS98 | R336G - E457T |
| DS99 | R336N - E457T - A397D |
| DS100 | R336N - E457T |
| DS101 | R336D - E457T - A397D |
| DS102 | R336D - E457T |
| DS103 | R454A - E457T |
| DS104 | E457T |
| DS105 | R454A - E457T - A397D |
| DS106 | R454A - E457T - A397K |
| DS107 | R454A - E457T - N474S |
| DS108 | D501X - E457T |
| DS109 | D501X - E457T - A397D |
| DS110 | D502X |
| DS111 | D502X - E457N |
| DS112 | D502X - E457TN - A397D |
| DS113 | D502X - E457S |
| DS114 | D502X - E457S - A397D |
| DS115 | D502X - E457T |
| DS116 | D502X - E457T - A397D |
| DS117 | D503X |
| DS118 | D5O3X - E457N |
| DS119 | D503X - E457TN - A397D |
| DS120 | D503X - E457S |
| DS121 | D503X - E457S - A397D |
| DS122 | D503X - E457T |
| DS123 | D5O3X - E457T - A397D |
| DS124 | R336N - R454A - E457N |
| DS125 | R336N - R454A - E457G |
| DS126 | R336N - E457G |
| DS127 | R336G - R454A - E457N |

In a particular embodiment, the variant is a chimeric construct of DNA polymerases of the polX family. “Chimeric construct” is understood to mean a chimeric enzyme formed by the addition, and in particular the fusion or the conjugation, of one or more predetermined sequences of an enzyme which is a member of the polX family as a replacement of one or more homologous sequences in the DNA polymerase variant considered.

Thus, the invention proposes a variant of the TdT of sequence SEQ ID No. 1 comprising, in addition to one or more point mutations in one and/or the other of the above positions, a substitution of the residues between the positions C378 to L406, or the functionally equivalent residues, by the residues H363 to C390 of the polymerase Polµ of sequence SEQ ID No. 2, or the functionally equivalent residues.

Alternatively or additionally, variants which are the subject matters of the present invention can have a deletion of one or more successive amino acid residues at the N-terminal end. These deletions can target in particular one or more enzymatic domains involved in the bond with other proteins and/or involved in the cellular localization. For example, the polypeptide sequence of the TdT comprises at the N-terminal end a BRCT domain of interaction with other proteins such as Ku70/80 and a nuclear localization domain (NLS).

In a particular embodiment of the present invention, the variant is a variant of the TdT of sequence SEQ ID No. 1 having, in addition to one or more of the mutations described above, a deletion of the residues 1-129 corresponding to the N-terminal end of the wild-type TdT.

In certain particular cases, the mutagenesis strategies can be guided by known information such as the sequences of natural variants, the sequence comparison with bound proteins, physical properties, the study of a three-dimensional structure or computer simulations involving such entities.

The present invention relates to a nucleic acid coding for a variant of a DNA polymerase of the polX family capable of synthesizing a nucleic acid molecule without a template strand according to the present invention. The present invention also relates to an expression cassette of a nucleic acid according to the present invention. The invention further relates to a vector comprising a nucleic acid or an expression cassette according to the present invention. The vector can be selected from a plasmid or a viral vector.

The nucleic acid coding for the DNA polymerase variant can be DNA (cDNA or gDNA), RNA, a mixture of the two. It can be in single-strand form or in duplex form or a mixture of the two forms. It can comprise modified nucleotides comprising, for example, a modified bond, a modified purine or pyrimidine base, or a modified sugar. It can be prepared by any of the methods known to the person skilled in the art, including chemical synthesis, recombination, mutagenesis, etc...

The expression cassette comprises all the elements necessary for the expression of the variant of a DNA polymerase of a polX family capable of synthesizing a nucleic acid molecule without a template strand according to the present invention, in particular the elements necessary for transcription and translation in the host cell. The host cell can be prokaryotic or eukaryotic. In particular, the expression cassette comprises a promoter and a terminator, optionally an amplifier. The promoter can be prokaryotic or eukaryotic. The following are examples of preferred prokaryotic promoters: Lacl, LacZ, pLacT, ptac, pARA, pBAD, the bacteriophage T3 or T7 RNA polymerase promoters, the polyhydrin promoter, the lambda phage PR or PL promoter. The following are examples of preferred eukaryotic promoters: the early CMV promoter, the HSV thymidine kinase promoter, the early or late SV40 promoter, the murine murine metallothionein-L promoter, and LTR regions of certain retroviruses. In general, for the selection of an appropriate promoter, the person skilled in the art can advantageously refer to the work by Sambrook et al. (1989) or to the techniques described by Fuller et al. (1996; Immunology in Current Protocols in Molecular Biology).

The present invention relates to a vector carrying a nucleic acid or an expression cassette coding for a variant of a DNA polymerase of the polX family capable of synthesizing a nucleic acid molecule without a template strand according to the present invention. The vector is preferably an expression vector, that is to say it comprises the elements necessary for the expression of the variant in the host cell. The host cell can be a prokaryote, for example, E. coli, or a eukaryote. The eukaryote can be a lower eukaryote such as a yeast (for example, P. pastoris or K. lactis) or a fungus (for example, of the Aspergillus genus) or a higher eukaryote such as an insect cell (Sf9 or Sf21, for example), a mammalian cell or a plant cell. The cell can be a mammalian cell, for example, COS (green monkey cell line) (for example, COS 1 (ATCC CRL-1650), COS 7 (ATCC CRL-1651), CHO (US 4,889,803; US 5,047,335, CHO-K1 (ATCC CCL-61)), murine cells and human cells. In a particular embodiment, the cell is non-human and non-embryonic. The vector can be a plasmid, a phage, a phagemid, a cosmid, a virus, a YAC, a BAC, an Agrobacterium pTi plasmid, etc... The vector can preferably comprise one or more elements selected from a replication origin, a multiple cloning site and a selection gene. In a preferred embodiment, the vector is a plasmid. The following are non-exhaustive examples of prokaryotic vectors: pQE70, pQE60, pQE-9 (Qiagen), pbs, pD10, phagescript, psiX174, pbluescrip SK, pbsks, pNH8A, pNH16A, pNH18A, pNH46A (Stratagene); ptrc99a, pKK223-3, pKK233-3, pDR540, pBR322, and pRIT5 (Pharmacia), pET (Novagen). The following are non-exhaustive examples of eukaryotic vectors: pWLNEO, pSV2CAT, pPICZ, pcDNA3.1 (+) Hyg (Invitrogen), pOG44, pXT1, pSG (Strategene); pSVK3, pBPV, pCI-neo (Stratagene), pMSG, pSVL (Pharmacia); and pQE-30 (QLAexpress). The viral vectors can be in a non-exhaustive manner adenoviruses, AAV, HSV, lentiviruses, etc... Preferably, the expression vector is a plasmid or a viral vector.

The sequence coding for the variant according to the present invention may or may not comprise a signal peptide. In the case in which it does not comprise a signal peptide, a methionine can optionally be added to the N-terminal end. In another alternative, a heterologous signal peptide can be introduced. This heterologous signal peptide can be derived from a prokaryote such as E. coli or from a eukaryote, in particular a mammalian cell, an insect cell, or a yeast.

The present invention relates to the use of a polynucleotide, of an expression cassette or of a vector according to the present invention for transforming or transfecting a cell. The present invention relates to a host cell comprising a nucleic acid, an expression cassette or a vector coding for a variant of a polymerase DNA of the polX family capable of synthesizing a nucleic acid molecule without a template strand and to its use for producing a variant of a DNA polymerase of the polX family capable of synthesizing a nucleic acid molecule without a recombinant template strand according to the present invention. The term “host cell” encompasses the daughter cells resulting from the culture or from the growth of this cell. In a particular embodiment, the cell is non-human and non-embryonic. The present invention also relates to a method for producing a variant of a DNA polymerase of the polX family capable of synthesizing a nucleic acid molecule without a recombinant template strand according to the present invention, comprising the transformation or transfection of a cell by a polynucleotide, an expression cassette or a vector according to the present invention; the culturing of the transfected/transformed cell; and the harvesting of the variant of a DNA polymerase of the polX family capable of synthesizing a nucleic acid molecule without a template strand produced by the cell. In an alternative embodiment, a method for producing a variant of a DNA polymerase of the polX family capable of synthesizing a nucleic acid molecule without recombinant template strand according to the present invention comprises the provision of a cell comprising a polynucleotide, an expression cassette or a vector according to the invention; the culturing of the transfected/transformed cell; and the harvesting of the variant of a DNA polymerase of the polX family capable of synthesizing a nucleic acid molecule without a template strand produced by the cell. In particular, the cell can be transformed/transfected in a transient or stable manner by the nucleic acid coding for the variant. This nucleic acid can be contained in the cell in the form of an episome or in chromosomal form. The methods for producing recombinant proteins are well known to the person skilled in the art. For example, it is possible to cite the specific procedures described in US 5,004,689, EP 446 582, Wang et al. (Sci. Sin. B 24:1076-1084, 1994 and Nature 295, page 503) for production in E. coli, and JAMES et al. (Protein Science (1996), 5:331-340) for production in mammalian cells.

The DNA polymerase variants according to the present invention are particularly advantageous for the synthesis of nucleic acids without a template strand. More particularly, the variants according to the invention have an enlarged catalytic pocket which is particularly suitable for the synthesis of nucleic acid by means of modified nucleotides exhibiting greater steric hindrance than the natural nucleotides. The variants according to the invention can in particular make it possible to incorporate modified nucleotides such as those described in the application WO2016/034807 in a nucleic acid strand.

The kinetics of incorporations of DNA polymerase variants and in particular of the variants of the TdT according to the invention, presenting the mutations or the combinations of specific mutations described above, are greatly improved in comparison to the kinetics of incorporation of a wild-type DNA polymerase. These variants can advantageously be used in the context of a high-performance enzymatic DNA synthesis method.

Thus, the invention also relates to a use of a variant of a DNA polymerase of the polX family according to the present invention for synthesizing a nucleic acid molecule without a template strand, from 3′-OH modified nucleotides, and in particular those described in the application WO2016034807.

The invention also relates to a method for the enzymatic synthesis of a nucleic acid molecule without a template strand, according to which a primer strand is brought in contact with at least one nucleotide, preferably a 3′-OH modified nucleotide, in the presence of a variant of a DNA polymerase of the polX family according to the invention.

Advantageously, the variants according to the invention can be used to carry out the synthesis method described in the application WO2015/159023.

The invention also relates to a kit for the enzymatic synthesis of a nucleic acid molecule without a template strand, comprising at least one variant of a DNA polymerase of the polX family according to the invention, nucleotides, preferably 3′-OH modified nucleotides, and optionally at least one nucleotide primer.

All the references cited in this description are incorporated by reference in the present application. Other features and advantages of the invention will become clearer upon reading the following examples which are of course for illustration and non-limiting.

### EXAMPLES

### Example 1- Generation, Production and Purification of DNA Polymerase Variants of the polX Family According to the Invention

### Generation of the Producer Strains

The truncated gene of the murine TdT was generated from the plasmid pET28b, the construction of which is described in [Boulé et al., 1998, Mol. Biotechnol., 10, 199-208]. The corresponding sequence SEQ ID No. 3 (corresponding to SEQ ID No. 1 truncated by the first 120 amino acids) was amplified using the following primers:
❖ T7-pro: TAATACGACTCACTATAGGG (SEQ ID No. 7)
❖ T7-ter: GCTAGTTATTGCTCAGCGG (SEQ ID No. 8)
according to the usual PCR amplification and molecular biology techniques. It was cloned in a plasmid pET32 to yield the vector pET32-SEQ ID No. 3.

The plasmid pET32-SEQ ID No. 3 was first sequenced, and then transformed in the commercial E. coli strains BL21 (DE3) (Novagen). The colonies that were capable of growing in kanamycin/chloramphenicol petri dishes were isolated and labeled Ec-SEQ ID No. 3.

### Generation of the Variants

The vector pET32-SEQ ID No. 3 was used as starting vector. Primers comprising the point mutation (or in some cases the point mutations if they are sufficiently close) were generated from the online tool of Agilent: (http://www.genomics.agilent.com/primerDesignProgram.jsp)

The QuickChange II (Agilent) kit was used to generate the plasmids of the variants comprising the desired mutation(s). The mutagenesis protocol given by the manufacturer was scrupulously respected in order to obtain a plasma pET32-DSi (i is the number of the variant in question given in table 1). At the end of the procedure, the plasmid pET32-DSx was first sequenced, then transformed in the commercial E. coli strains BL21 (DE3) (Novagen). The colonies that were capable of growing in kanamycin/chloramphenicol petri dishes were isolated and labeled Ec-DSx.

### Production

The cells Ec- SEQ ID No. 3 and Ec-DSx were precultured in 250 mL Erlenmeyer flasks containing 50 mL of LB medium to which appropriate quantities of kanamycin and chloramphenicol were added. The culture was incubated at 37° C. under stirring overnight. The preculture was then used to inoculate a 5 L Erlenmeyer flask containing 2 L of LB medium with the addition of appropriate quantities of kanamycin and chloramphenicol. The starting optical density (OD) was 0.01. The culture was incubated at 37° C. under stirring. The OD was measured regularly until a value between 0.6 and 0.9 was reached. Once this value was reached, 1 mL of isopropyl β-D-1-thiogalactopyranoside 1 M was added to the culture medium. The culture was incubated again at 37° C. until the next day. The cells were then harvested by centrifugation without exceeding 5,000 rpm. The different pellets obtained were collected to form a single pellet during the washing with the lysis buffer (20 mM Tris-HCl, pH 8.3, 0.5 M NaCl). The cell pellet was frozen at -20° C. It can be stored in this way for several months.

### Extraction

The cell pellet frozen during the preceding step was thawed in a water bath heated at 25 to 37° C. Once the thawing was completed, the cell pellet was resuspended in approximately 100 mL of lysis buffer. Particular attention was paid to the resuspension which must lead to a very homogeneous solution and in particular to complete absence of aggregates. Thus resuspended, the cells were lysed using a French press at a pressure of 14,000 psi. The lysate collected was centrifuged at high speed, 10,000 g for 1 h to 1 h 30. The centrifugate was filtered through a 0.2 µM filter and collected in a tube of sufficient volume.

### Purification

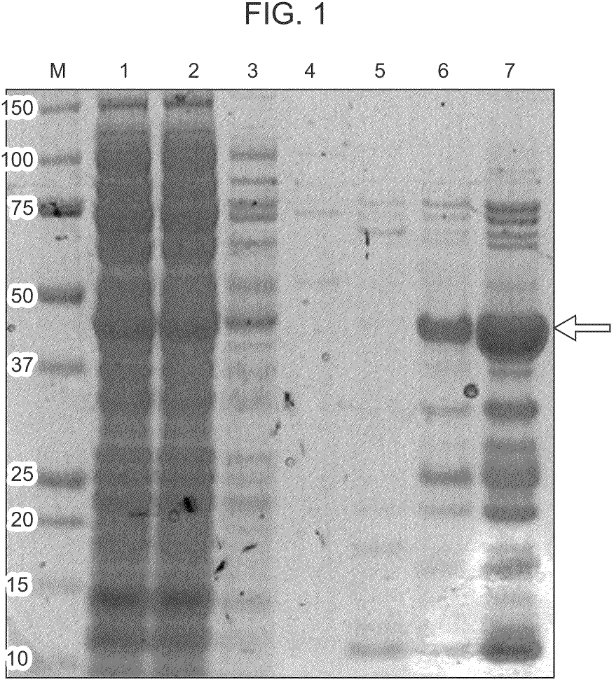

The TdT was purified on an affinity column. 5 mL His-Trap Crude (GE Life Sciences) columns were used with peristaltic pumps (Peristaltic Pump - MINIPULS® Evolution, Gilson). In a first step, the column was equilibrated using 2 to 3 CV (column volume) of lysis buffer. The centrifugate of the preceding step was then loaded onto the column at a rate of approximately 0.5 to 5 mL/min. Once all the centrifugate was loaded, the column was washed using 3 CV of lysis buffer, then 3 CV of washing buffer (20 mM Tris-HCl, pH 8.3, 0.5 M NaCl, 60 mM imidazole). At the end of this step, the elution buffer (20 mM Tris-HCl, pH 8.3, 0.5 M NaCl, 1 M imidazole) was injected in the column at approximately 0.5 to 1 mL/min for a total volume of 3 CV. During the entire elution phase, the outflow of the column was collected in 1 mL fractions. These fractions were analyzed by SDS-PAGE, in order to determine which fractions contain the elution peak. Once the fractions were determined, they were pooled to a form a single fraction and dialyzed against the dialysis buffer (20 mM Tris-HCl, pH 6.8, 200 mM NaCl, 50 mM MgOAc, 100 mM [NH₄]₂SO₄. The TdT was then concentrated (Amicon Ultra-30 centrifuge filters, Merk Millipore) to a final concentration of 5 to 15 mg/mL. The concentrated TdT was frozen at -20° C. for long-term storage after the addition of 50% glycerol. Throughout the entire purification phase, aliquots of different samples were collected (approximately 5 µL) for an SDS-PAGE gel analysis, the results of which are presented in FIG. 1.

### Example 2 — Alignment of Sequences Between Different Polymerases of the polX Family Capable of Being Used for the Creation of Variants According to the Invention

Different DNA polymerases of the polX family were aligned using the online alignment software Mutalin (http://multalin.toulouse.inra.fr/multalin/multalin .html, accessed on Apr. 4, 2016).

TABLE 2

Aligned sequences

| Identifier | DNA polymerase | Species | Length |
|---|---|---|---|
| Q9NP87 | Pol µ (SEQ ID No. 2) | Homo sapiens | 494 |
| H2QUI0 | Pol µ (SEQ ID No. 9) | Pan troglodytes | 494 |
| Q924W4 | Pol µ (SEQ ID No. 10) | Mus musculus | 496 |
| F1P657 | TdT (SEQ ID No. 11) | Canis lupus familiaris | 509 |
| Q3UZ80 | TdT (SEQ ID No. 1) | Mus musculus | 510 |
| P36195 | TdT (SEQ ID No. 12) | Gallus gallus | 506 |
| P04053 | TdT (SEQ ID No. 13) | Homo sapiens | 509 |

The alignments obtained are presented in FIG. 2.

### Example 3 - Study of the Activity of the Variants in the Presence of Non-Natural Substrates

The activity of different variants according to the invention was determined by the following test. The results were compared to those obtained with the natural enzyme from which each of the variants is derived.

### Activity Test

TABLE 3

Reaction mixture

| | | |
|---|---|---|
| Reagent | Concentration | Volume |
| H₂O | - | 15 µL |
| Primer | 500 nM | 2.5 µL |
| Buffer | 10x | 2.5 µL |
| Modified nucleotide | 250 µM | 2.5 µL |
| Enzyme | 20 µM | 2.5 µL |

The primer used, of sequence 5′-AAAAAAAAAAGGGG-3′ (SEQ ID No. 14), was radioactively labeled at 5′ beforehand by means of a standard labeling protocol involving the enzyme PNK (NEB) and the use of radioactive ATP (PerkinElmer).

The buffer 10x consisting of 250 mM Tris-HCl pH 7.2, 80 mM MgCl₂, 3.3 mM ZnSO₄ was used.

The modified nucleotides used are 3′-O-amino-2′,3′-dideoxynucleotides-5′-triphosphate (ONH2, Firebird Biosciences) or 3′-biot-EDA-2′,3′-dideoxynucleotides-5′-triphosphate (Biot-EDA, Jena Biosciences), such as 3′-O-amino-2′,3′-dideoxyadenosine-5′-triphosphate or 3′-biot-EDA-2′,3′-dideoxyadenosine-5′-triphosphate, for example. The 3′-O-amino group is a group of larger volume bound to the 3′-OH end. The 3′-biot-EDA group is an extremely large-volume and inflexible group bound to the 3′-OH end.

The performances of incorporation of a modified nucleotide given by the variants produced by the variants listed in table 1 were evaluated in comparison to the natural TdT (SEQ ID No. 3) by carrying out simultaneous activity tests for which only the enzyme varies.

The reagents were added in the order given in table 3 above and then incubated at 37° C. for 90 min. The reaction was then stopped by the addition of formamide blue (formamide 100%, 1 to 5 mg of bromophenol blue; Simga)

### Gel and Radiography

A 16% polyacrylamide denaturing gel (Biorad) was used for the analysis of the preceding activity test. The gel was first poured and allowed to polymerize. Then it was mounted on an electrophoresis tank having appropriate dimensions, filled with TBE buffer (Sigma). The different samples were loaded directly on the gel without pretreatment.

The gel was then subjected to a potential difference of 500 to 2000 V for 3 to 6 hours. Once the migration was satisfactory, the gel was dismounted and then transferred to an incubation cassette. The phosphor screen (Amersham) was used for 10 to 60 min for imaging by means of a Typhoon instrument (GE Life Sciences) which was parameterized beforehand with an appropriate detection mode.

### Results

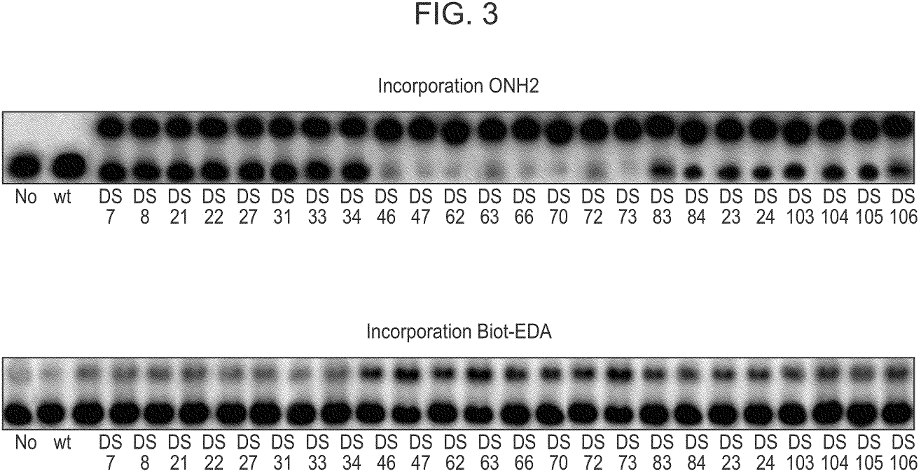

The comparative results of the two enzymes used are presented in FIG. 3.

More precisely, on the first gel (ONH2 incorporation), the natural TdT (wt column) is incapable of incorporating the 3′-O-amino-2′,3′-dideoxyadenosine-5′-triphosphate modified nucleotides as shown by the comparison with the negative control (No column).

Among the different variants, 3 different groups can be observed:

A first group of variants (columns DS7 to DS34) is capable of approximately 50% incorporation.

A second group of variants (columns DS46 to DS73) is capable of more than 95%, sometimes more than 98% incorporation.

A third group of variants (columns DS83 to DS106) is capable of 60 to 80% incorporation.

On the second gel (Biot-EDA incorporation), the natural TdT (wt column) is also incapable of incorporating the 3′-biot-EDA-2′,3′-dideoxyadenosine-5′-triphosphate modified nucleotides, as shown by the comparison with the negative control (No column).

Among the different variants, 3 different groups can be observed:

A first group of variants (columns DS7 to DS34) is capable of approximately 5 to 10% incorporation.

A second group of variants (columns DS46 to DS73) is capable of more than 30%, sometimes more than 40% incorporation.

A third group of variants (columns DS83 to DS106) is capable of 10 to 25% incorporation.

These results confirm that, in contrast to the wild-type enzyme, the variants of the TdT according to the invention are all capable of using modified nucleotides, in particular 3′-OH modified nucleotides, as a substrate. Particularly advantageously, certain variants have very high incorporation rates and this even in the presence of nucleotides carrying modifications which tend to result in a very large increase in the steric hindrance of said nucleotide.

### Example 4 - Study of the Kinetics of the Variants According to the Invention

A mutant having the combination of substitutions R336N - R454A - E457N (DS124) was generated and produced according to the preceding example 1.

### Activity Test

In the activity test, the enzymes are brought in the presence of ONH2 modified nucleotides and incubated at 37° C. for different times. The reactions are stopped in order to observe the kinetics of incorporation of DS 124 and to compare it with the kinetics of the natural WT enzyme (SEQ ID No. 3).

TABLE 4

Reaction mixture

| | | |
|---|---|---|
| Reagent | Concentration | Volume |
| H₂O | - | 15 µL |
| Buffer | 10x | 2.5 µL |
| Nucleotides | 2.5 µM | 2.5 µL |
| Enzyme | 80 µM | 2.5 µL |
| Primer | 1 µM | 2.5 µL |

The primer and the buffer used are in accordance with example 3.

The modified nucleotides used are 3′-O-amino-2′,3′-dideoxynucleotides-5′-triphosphate (ONH2, Firebird Biosciences): 3′-O-amino-2′,3′-dideoxyguanosine-5′-triphosphate, 3′-O-amino-2′,3′-dideoxycytidine-5′-triphosphate and 3′-O-amino-2′,3′-dideoxythymidine-5′-triphosphate. The 3′-O-amino group is a larger volume group bound to the 3′-OH end.

The performances of incorporation of the mixture of nucleotides by the enzyme DS 124 were evaluated by carrying out activity tests for which premixes containing all the reagents (added in the order of table 4) except for the primer were prepared. They are distributed in different reaction wells. At the initial time t = 0, the primer is added to all the wells simultaneously. At the different times t = 2 min, t = 5 min, t = 10 min, t = 15 min, t = 30 min and t = 90 min, the reaction is stopped by the addition of formamide blue (formamide 100%, 1 to 5 mg of bromophenol blue; Simga).

### Gel and Radiography

The analysis of the activity test is carried out by migration of the different samples in a polyacrylamide gel according to the protocol described in example 3.

### Results

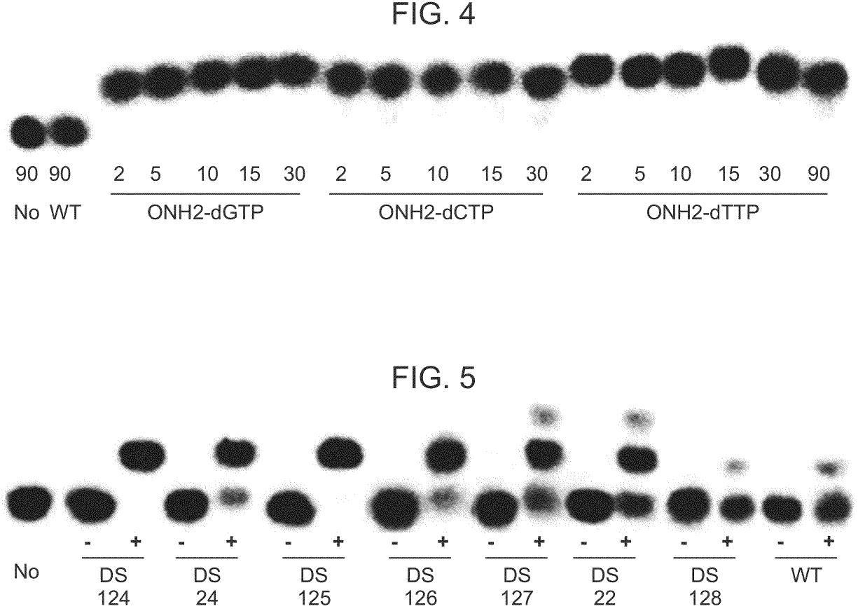

The comparative results of the two enzymes (DS 124 and WT) are presented in FIG. 4.

More precisely, on this gel, the negative control (No column) gives the expected size of the primer used when it has not been elongated, that is to say when there has been no incorporation of nucleotides. The natural TdT (WT column) is not capable of incorporating the modified nucleotides (here ONH2-dGTP): a band can be observed at the same level as that of the No column.

For all the nucleotides tested and for all the times from 90 min (used here as a positive control) to 2 min, corresponding to a reduction in the incubation time by a factor of 45, the variant DS124 is capable of incorporating the modified nucleotides with an apparent effectiveness of 100%.

These results confirm that the variants of the TdT according to the invention are capable of incorporation performances much higher than those of the natural TdT, in terms of both incorporation effectiveness and rapidity of incorporation. The kinetics of the variants of the TdT according to the invention are greatly improved by the mutations or combinations of specific mutations described by the present invention.

### Example 5 - Study of the Specificity of the Variants According to the Invention

The mutants having a substitution combination according to table 5 below were generated and produced according to example 1.

TABLE 5

List of the enzymatic variants used

| | |
|---|---|
| # | Combinations of mutations |
| DS124 | R336N - R454A - E457N |
| DS24 | R336N - E457N |
| DS125 | R336N - R454A - E457G |
| DS126 | R336N - E457G |
| DS127 | R336G - R454A - E457N |
| DS22 | R336G - E457N |
| DS128 | R336A - R454A - E457G |
| WT | SEQ ID No. 3 |

### Activity Test

In this activity test, the different variants were put in the presence of a mixture of natural nucleotides and of highly concentrated modified nucleotides. The concentration of the enzyme is also increased in order to shorten the incubation time and to achieve a quantitative addition (compare example 4).

The activity of different variants generated was determined by the following test:

Each variant is tested according to two conditions: (1) in the absence of nucleotides (replaced by H₂O) or (2) in the presence of the mixture of nucleotides. The results of the different variants are compared to one another. A control sample was added; it contained neither nucleotide nor enzyme (which were replaced by H₂O).

TABLE 6

Reaction mixture

| | | |
|---|---|---|
| Reagent | Concentration | Volume |
| H₂O | - | 15 µL |
| Primer | 1 µM | 2.5 µL |
| Buffer | 10x | 2.5 µL |
| Mixture nucleotides (10:90) | 2.5 µM | 2.5 µL |
| Enzyme | 80 µM | 2.5 µL |

The primer and the buffer used are identical to example 3.

When present, the mixture of nucleotides consists of natural 2′-deoxynucleotide 5′-triphosphate nucleotides (Nuc, Sigma-Aldrich) such as 2′-deoxyguanosine 5′-triphosphate (dGTP) and of 3′-O-amino-2′,3′-dideoxynucleotides-5′-triphosphate modified nucleotides (ONH2, Firebird Biosciences) such as 3′-O-amino-2′,3′-dideoxyguanosine-5′-triphosphate, for example. The 3′-O amino group of larger volume bound to the 3′-OH end. The mixture consists of 90% ONH2-dGTP modified nucleotides and 10% of natural dGTP nucleotides.

The incorporation performances of the mixture of nucleotides by the variants listed in table 5 compared to one another were evaluated by carrying out simultaneous activity tests, for which only the enzyme varies.

The reagents were added in the order given in table 6 above, and then incubated at 37° C. for 15 min. The reaction was then stopped by the addition of formamide blue (formamide 100%, 1 to 5 mg of bromophenol blue; Simga).

### Gel and Radiography

The analysis of the activity test was carried out by migration of the different samples in a polyacrylamide gel according to the protocol described in example 3.

### Results

The comparative results of the enzymes used are presented in FIG. 5.

More precisely, on this gel, the negative control (No column) gives the expected size of the primer used when it has not been elongated, that is to say when there has been no incorporation of nucleotides. The following samples are used in pairs, each pair corresponding to the same enzymatic variant tested under the two conditions: in the absence and in the presence of nucleotides (in the form of a mixture when they are present).

Among the different variants tested, 3 different groups can be observed:

The first group is the variant DS 128, which constitutes a negative control. This variant has extremely low rates of incorporation of the nucleotides: 5% to 10% incorporation is observed when the mixture of nucleotides is present; this corresponds to the proportion of natural nucleotides present in the mixture.

The second group consists of the variants DS 127 and DS22. These variants have high rates of incorporation of the nucleotides: 50% to 60% of incorporation is observed when the mixture of nucleotides is present. In this case, a band of further addition corresponding to the successive incorporation of two nucleotides is always observed for these two variants. The intensity of this band corresponds to the proportion of natural nucleotides present in the mixture of nucleotides.

The last group consists of the variants DS 124, DS24, DS 125 and DS 126. These variants have extremely high rates of incorporation of the nucleotides: 80% to 100% for DS124 and DS 125, when the mixture of nucleotides is present. In this case, no band of further addition is present. In the case of the variants DS24 and DS 126, the proportion of non-incorporation is similar to the proportion of natural nucleotides present in the mixture.

These results confirm that the variants of the TdT according to the invention are capable of preferentially using the modified nucleotides among a mixture of modified nucleotides and natural nucleotides. In a particularly advantageous manner, these variants have extremely high rates of incorporation of the modified nucleotides and are capable of discriminating the natural nucleotides in such a manner as not to incorporate them and thus greatly improve the quality of the DNA to be synthesized by avoiding the further additions.

### Example 6 - Example of the Synthesis of a DNA Strand Without a Template Strand

A variant of TdT having the combination of substitutions R336N - R454A - E457G (DS125) was generated and produced according to example 1.

The variant DS125 is used to synthesize the sequence: 5′-GTACGCTAGT-3′ (SEQ ID No. 15) after the primer of sequence 5′-AAAAAAAAAAGGGG-3′ (SEQ ID No. 14). The primer was radioactively labeled at 5′ beforehand by means of a standard labeling protocol involving the enzyme PNK (NEB) and the use of radioactive ATP (PerkinElmer).

The primer is bound to a solid support by interaction with a capture fragment of complementary sequence: 5′-CCTTTTTTTTTT-3′ (SEQ ID No. 16). The capture fragment possesses at its 3′ end a group which enables it to react covalently with a reaction group bound to a surface. For example, this group can be NH2, the reaction group N-hydroxysuccinimide, and the surface of a magnetic bead (Dynabeads, Thermofisher). The interaction of the primer with the capture fragment is carried out under standard DNA fragment hybridization conditions.

The modified nucleotides used are 3′-O-amino-2′,3′-dideoxynucleotides-5′-triphosphate (ONH2, Firebird Biosciences) such as 3′-O-amino-2′,3′-dideoxyguanosine-5′-triphosphate, 3′-O-amino-2′,3′-dideoxycytidine-5′-triphosphate, 3′-O-amino-2′,3′-dideoxythymidine-5′-triphosphate or 3′-O-amino-2′,3′-dideoxyadenosine-5′-triphosphate. The 3′-O-amino group is a larger volume group bound to the 3′-OH end.

### Synthesis

TABLE 7

Reaction mixture

| | | |
|---|---|---|
| Reagent | Concentration | Volume |
| H₂O | - | 210 µL |
| Buffer | 10x | 70 µL |
| Nucleotides | 2.5 µM | 35 µL |
| Enzyme | 80 µM | 35 µL |
| Primer on solid support | 1 µM | - |

The buffer 10x consisting of 250 mM Tris-HCl pH 7.2, 80 mM MgCl2, 3.3 mM ZnSO4 was used.

The washing buffer L used consists of Tris-HCl 25 mM at pH 7.2.

The deprotection buffer D used consists of sodium acetate 50 mM, pH 5.5 in the presence of 10 mM MgCl2.

Before the start of the synthesis, the beads constituting the solid support on which the primers were hybridized for a total equivalent quantity of primer of 35 pmol were washed several times with the buffer L. After these washings, the beads were held on a magnet, and the supernatant was removed in its entirety.

Several premixes consisting of different reagents added in the order of table 7 were prepared. Each of these premixes contains different nucleotides according to table 8 below.

TABLE 8

Composition of the premixes

| Premix number | Nucleotide of the premix |
|---|---|
| 1 | G |
| 2 | T |
| 3 | A |
| 4 | C |
| 5 | G |
| 6 | C |
| 7 | T |
| 8 | A |
| 9 | G |
| 10 | T |

The synthesis starts when the premix 1 is added to the beads which have been washed beforehand and freed from their supernatant. The synthesis steps according to table 9 below follow after one another, in order to produce the new sequence 5′-GTACGCTAGT-3′.

TABLE 9

Step of the method of synthesis of a DNA strand without a template strand

| | | | |
|---|---|---|---|
| Steps | Action | Volume | Duration |
| Elongation 1 | Addition premix 1 | 350 µL | 15 min |
| Sampling 1 | Sampling | 5 µL | < 1 min |
| 1st Washing 1 | Addition buffer L | 350 µL | 5 min |
| 1st Deprotection 1 | Additional buffer D | 350 µL | 15 min |
| 2nd Deprotection 1 | Additional buffer D | 350 µL | 15 min |
| 2nd Washing 1 | Addition buffer L | 350 µL | 5 min |
| Elongation 2 | Addition premix 2 | 350 µL | 15 min |
| Sampling 2 | Sampling | 5 µL | < 1 min |
| 1st Washing 2 | Addition buffer L | 350 µL | 5 min |
| 1st Deprotection 2 | Addition buffer D | 350 µL | 15 min |
| 2nd Deprotection 2 | Addition buffer D | 350 µL | 15 min |
| 2nd Washing 2 | Addition buffer L | 350 µL | 5 min |
| Elongation 3 | Addition premix 3 | 350 µL | 15 min |
| Sampling 3 | Sampling | 5 µL | < 1 min |
| 1st Washing 3 | Addition buffer L | 350 µL | 5 min |
| 1st Deprotection 3 | Addition buffer D | 350 µL | 15 min |
| 2nd Deprotection 3 | Addition buffer D | 350 µL | 15 min |
| 2nd Washing 3 | Addition buffer L | 350 µL | 5 min |
| Elongation 4 | Addition premix 4 | 350 µL | 15 min |
| Sampling 4 | Sampling | 5 µL | < 1 min |
| 1st Washing 4 | Addition buffer L | 350 µL | 5 min |
| 1st Deprotection 4 | Addition buffer D | 350 µL | 15 min |
| 2nd Deprotection 4 | Addition buffer D | 350 µL | 15 min |
| 2nd Washing 4 | Addition buffer L | 350 µL | 5 min |
| Elongation 5 | Addition premix 5 | 350 µL | 15 min |
| Sampling 5 | Sampling | 5 µL | < 1 min |
| 1st Washing 5 | Addition buffer L | 350 µL | 5 min |
| 1st Deprotection 5 | Addition buffer D | 350 µL | 15 min |
| 2nd Deprotection 5 | Addition buffer D | 350 µL | 15 min |
| 2nd Washing 5 | Addition buffer L | 350 µL | 5 min |
| Elongation 6 | Addition premix 6 | 350 µL | 15 min |
| Sampling 6 | Sampling | 5 µL | < 1 min |
| 1st Washing 6 | Addition buffer L | 350 µL | 5 min |
| 1st Deprotection 6 | Addition buffer D | 350 µL | 15 min |
| 2nd Deprotection 6 | Addition buffer D | 350 µL | 15 min |
| 2nd Washing 6 | Addition buffer L | 350 µL | 5 min |
| Elongation 7 | Addition premix 7 | 350 µL | 15 min |
| Sampling 7 | Sampling | 5 µL | < 1 min |
| 1st Washing 7 | Addition buffer L | 350 µL | 5 min |
| 1st Deprotection 7 | Addition buffer D | 350 µL | 15 min |
| 2nd Deprotection 7 | Addition buffer D | 350 µL | 15 min |
| 2nd Washing 7 | Addition buffer L | 350 µL | 5 min |
| Elongation 8 | Addition premix 8 | 350 µL | 15 min |
| Sampling 8 | Sampling | 5 µL | < 1 min |
| 1st Washing 8 | Addition buffer L | 350 µL | 5 min |
| 1st Deprotection 8 | Addition buffer D | 350 µL | 15 min |
| 2nd Deprotection 8 | Addition buffer D | 350 µL | 15 min |
| 2nd Washing 8 | Addition buffer L | 350 µL | 5 min |
| Elongation 9 | Addition premix 9 | 350 µL | 15 min |
| Sampling 9 | Sampling | 5 µL | < 1 min |
| 1st Washing 9 | Addition buffer L | 350 µL | 5 min |
| 1st Deprotection 9 | Addition buffer D | 350 µL | 15 min |
| 2nd Deprotection 9 | Addition buffer D | 350 µL | 15 min |
| 2nd Washing 9 | Addition buffer L | 350 µL | 5 min |
| Elongation 10 | Addition premix 10 | 350 µL | 15 min |
| Sampling 10 | Sampling | 5 µL | < 1 min |

Between each step, except for the sampling step, the beads are collected by means of a magnet, and the supernatant is removed in its entirety.

Each sample is added to a solution of 15 µL of formamide blue (formamide 100%, 1 to 5 mg of bromophenol blue; Simga) in order to stop the reaction and prepare the analysis.

### Gel and Radiography

The analysis of the activity test is carried out by migration of the different samples in a polyacrylamide gel according to the protocol described in example 3.

### Results

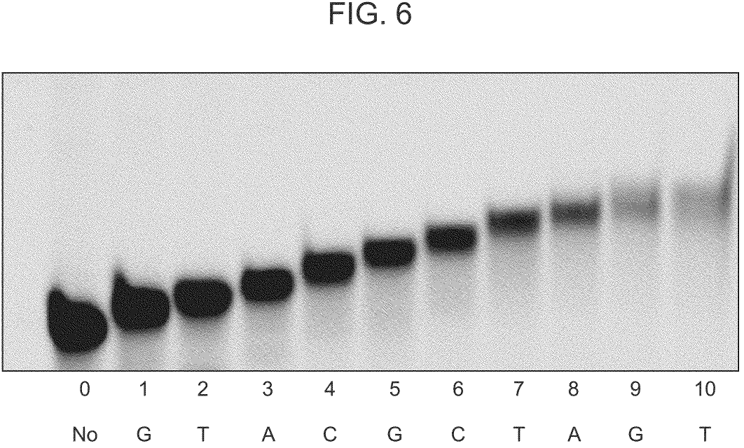

The results of this synthesis are presented in FIG. 6.

Column 0 (No, no nucleotides) gives the expected size of the primer used, when it has not been elongated, that is to say when there has been no incorporation of nucleotides.

Columns 1 to 10 correspond to samples 1 to 10 during the synthesis. Each incorporation of nucleotides was carried out by the enzyme with maximum performance. No additional purification step is carried out.

A similar synthesis experiment was carried out with the natural TdT. The latter being incapable of incorporating modified nucleotides, it was not possible to synthesize the desired sequence.

1-24. (canceled)

25. A variant of a terminal deoxyribonucleotide transferase (TdT) comprising a substitution of alanine, valine, isoleucine, leucine, asparagine, cysteine, or glycine at amino acid position 336, wherein numbering of amino acid positions is determined by alignment with the sequence of SEQ ID NO: 1, wherein the sequence of the variant has at least 90 percent identity with the sequence of SEQ ID NO: 1, wherein the variant is capable of synthesizing a nucleic acid in absence of a template strand and incorporating a 3′-O-modified nucleoside triphosphate into a nucleic acid primer.

26. The variant of TdT of claim 25, wherein the variant of TdT further comprises a substitution of alanine, proline, or valine at amino acid position 454, wherein numbering of amino acid positions is determined by alignment with the sequence of SEQ ID NO: 1.

27. The variant of TdT of claim 25, wherein the variant of TdT further comprises a substitution of valine, leucine, asparagine, or glycine at amino acid position 457, wherein numbering of amino acid positions is determined by alignment with the sequence of SEQ ID NO: 1.

28. The variant of TdT of claim 25, wherein the variant of TdT comprises R336N-E457N substitutions, R336N-R454A-E457N substitutions, R336N-R454A-E457G substitutions, R336N-E457G substitutions, R336G-R454A-E457N substitutions, or R336G-E457N substitutions.

29. The variant of TdT of claim 25, wherein the variant of TdT is capable of synthesizing DNA or RNA strands.

30. The variant of TdT of claim 25, wherein the sequence of the variant of TdT has at least 95%, 96%, 97%, 98%, or 99% identity with the sequence of SEQ ID NO: 1.

31. The variant of TdT of claim 25, further comprising an N-terminal deletion of amino acid residues 1 to 129, wherein numbering of amino acid positions is determined by alignment with the sequence of SEQ ID NO: 1.

32. The variant of TdT of claim 25, further comprising a deletion of a BRCT domain or a nuclear localization domain (NLS) domain, or a combination thereof.

33. The variant of TdT of claim 25, further comprising a heterologous signal peptide.

34. The variant of TdT of claim 25, further comprising a polyhistidine tag.

35. A nucleic acid comprising a coding sequence encoding the variant of TdT of claim 25.

36. The nucleic acid of claim 35, wherein the variant of TdT comprises R336N-E457N substitutions, R336N-R454A-E457N substitutions, R336N-R454A-E457G substitutions, R336N-E457G substitutions, R336G-R454A-E457N substitutions, or R336G-E457N substitutions.

37. An expression cassette comprising and expressing the nucleic acid of claim 35.

38. The expression cassette of claim 37, wherein the variant of TdT comprises R336N-E457N substitutions, R336N-R454A-E457N substitutions, R336N-R454A-E457G substitutions, R336N-E457G substitutions, R336G-R454A-E457N substitutions, or R336G-E457N substitutions.

39. A vector comprising the expression cassette of claim 37.

40. The vector of claim 39, wherein the variant of TdT comprises R336N-E457N substitutions, R336N-R454A-E457N substitutions, R336N-R454A-E457G substitutions, R336N-E457G substitutions, R336G-R454A-E457N substitutions, or R336G-E457N substitutions.

41. A host cell comprising the vector of claim 39.

42. The host cell of claim 41, wherein the variant of TdT comprises R336N-E457N substitutions, R336N-R454A-E457N substitutions, R336N-R454A-E457G substitutions, R336N-E457G substitutions, R336G-R454A-E457N substitutions, or R336G-E457N substitutions.

43. A method of producing a variant of TdT, the method comprising culturing the host cell of claim 41 under conditions permitting expression of the nucleic acid encoding the variant of TdT, wherein the variant of TdT is produced.

44. The method of claim 43, further comprising recovering the variant of TdT from the culture medium or from said host cell.

45. The method of claim 43, wherein the variant of TdT comprises R336N-E457N substitutions, R336N-R454A-E457N substitutions, R336N-R454A-E457G substitutions, R336N-E457G substitutions, R336G-R454A-E457N substitutions, or R336G-E457N substitutions.

46. A method of using the variant of TdT of claim 25 for template-free synthesis of a nucleic acid molecule, the method comprising contacting a primer strand with a nucleotide and the variant of TdT of claim 25.

47. The method of claim 46, wherein the nucleotide comprises a modified 3′-hydroxyl group.

48. The method of claim 46, wherein the nucleic acid molecule is DNA or RNA.

49. The method of claim 46, wherein the variant of TdT comprises R336N-E457N substitutions, R336N-R454A-E457N substitutions, R336N-R454A-E457G substitutions, R336N-E457G substitutions, R336G-R454A-E457N substitutions, or R336G-E457N substitutions.

50. A kit for the enzymatic synthesis of nucleic acid molecules without using a template strand, the kit comprising the variant of TdT of claim 25 and nucleotides.

51. The kit of claim 50, wherein the nucleotides comprise modified 3′-hydroxyl groups.

52. The kit of claim 50, further comprising a primer.

53. The kit of claim 50, further comprising a divalent cation.

54. The kit of claim 50, wherein the variant of TdT comprises R336N-E457N substitutions, R336N-R454A-E457N substitutions, R336N-R454A-E457G substitutions, R336N-E457G substitutions, R336G-R454A-E457N substitutions, or R336G-E457N substitutions.


SEQUENCE LISTING (16 sequences)

SEQ ID NO: 1 - protein, 510 aa, synthetic construct
  REGION 1..510: note = synthetic sequence
MDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 2 - protein, 494 aa, synthetic construct
  REGION 1..494: note = synthetic sequence
MLPKRRRARVGSPSGDAASSTPPSTRFPGVAIYLVEPRMGRSRRAFLTGLARSKGFRVLDACSSEATHVVMEETSAEEAVSWQERRMAAAPPGCTPPALLDISWLTESLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLQGHDVDFLITHPKEGQEAGLLPRVMCRLQDQGLILYHQHQHSCCESPTRLAQQSHMDAFERSFCIFRLPQPPGAAVGGSTRPCPSWKAVRVDLVVAPVSQFPFALLGWTGSKLFQRELRRFSRKEKGLWLNSHGLFDPEQKTFFQAASEEDIFRHLGLEYLPPEQRNA

SEQ ID NO: 3 - protein, 401 aa, synthetic construct
  REGION 1..401: note = synthetic sequence
TMGSSHHHHHHSSGLVPRGSHMSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 4 - protein, 11 aa, synthetic construct
  VARIANT 1: note = The amino acid given in position 1 of the sequence can be M, I, V, or L
  VARIANT 2: note = The amino acid given in position 2 of the sequence can be T, A, M, or Q
  VARIANT 10: note = The amino acid given in position 10 of the sequence can be M, K, E, Q, L, S, P, R, or D
  VARIANT 11: note = The amino acid given in position 11 of the sequence can be T, I, M, F, K, V, Y, E, Q, H, S, R, or D
XXGGFRRGKXX

SEQ ID NO: 5 - protein, 13 aa, synthetic construct
  VARIANT 1: note = The amino acid given in position 1 of the sequence can be A, C, G, or S
  VARIANT 2: note = The amino acid given in position 2 of the sequence can be L, T, or R
  VARIANT 5: note = The amino acid given in position 5 of the sequence can be W or Y
  VARIANT 10: note = The amino acid given in position 10 of the sequence can be Q, L, H, F, Y, N, E, or D
  VARIANT 6: note = The amino acid given in position 6 of the sequence can be T, S, or I
  VARIANT 11: note = The amino acid given in position 11 of the sequence can be F or Y
XXLGXXGSRXXER

SEQ ID NO: 6 - protein, 11 aa, synthetic construct
  VARIANT 2: note = The amino acid given in position 2 of the sequence can be D, E, S, P, A, or K
  VARIANT 5: note = The amino acid given in position 5 of the sequence can be E, Q, P, Y, L, K, G, or N
  VARIANT 8: note = The amino acid given in position 8 of the sequence can be E, Q, D, H, or L
  VARIANT 4: note = The amino acid given in position 4 of the sequence can be I, L, M, V, A, or T
  VARIANT 7: note = The amino acid given in position 7 of the sequence can be W, S, V, E, R, Q, T, C, K, or H
LXYXXPXXRNA

SEQ ID NO: 7 - DNA, 20 bp, synthetic construct
  source 1..20: mol_type = other DNA
taatacgactcactataggg

SEQ ID NO: 8 - DNA, 19 bp, synthetic construct
  source 1..19: mol_type = other DNA
gctagttattgctcagcgg

SEQ ID NO: 9 - protein, 494 aa, Pan troglodytes
MLPKRRRARVGSPSGDAASSTPPSTRFPGVAIYLVEPRMGRSRRAFLTRLTRSKGFRVLDACSSEATHVVMEETSAEEAVSWQERRMAAAPPGCTPPALLDISWLTESLGAGQPVPVECRHRLEVAGPRKGPLSPAWMPAYVCQRPTPLTHHNTGLSEALETLAEAAGFEGSEGRLLTFCRAASVLKALPSPVTTLSQLQGLPHFGEHSSRVVQELLEHGVCEEVERVQRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLQGHDVDFLITHPKEGQEAGLLPRVMCRLQDQGLILYHQHQHSCWESPTRLAQQSHMDAFERSFCIFRLPQPPGAAVGGSTRPCPSWKAVRVDLVVAPVSQFPFALLGWTGSKLFQRELRRFSRKEKGLWLNSHGLFDPEQKTFFQAASEEDIFRHLGLEYLPPEQRNA

SEQ ID NO: 10 - protein, 496 aa, Mus musculus
MLPKRRRVRAGSPHSAVASSTPPSVVRFPDVAIYLAEPRMGRSRRAFLTRLARSKGFRVLDAYSSKVTHVVMEGTSAKEAICWQKNMDALPTGCPQPALLDISWFTESMAAGQPVREEGRHHLEVAEPRKEPPVSASMPAYACQRPSPLTHHNTLLSEALETLAEAAGFEANEGRLLSFSRADSVLKSLPCPVASLSQLHGLPYFGEHSTRVIQELLEHGTCEEVKQVRCSERYQTMKLFTQVFGVGVKTANRWYQEGLRTLDELREQPQRLTQQQKAGLQYYQDLSTPVRRADAEALQQLIEAAVRQTLPGATVTLTGGFRRGKLQGHDVDFLITHPEEGQEVGLLPKVMSCLQSQGLVLYHQYHRSHLADSAHNLRQRSSTMDAFERSFCILGLPQPQQAALAGALPPCPTWKAVRVDLVVTPSSQFPFALLGWTGSQFFERELRRFSRQEKGLWLNSHGLFDPEQKRVFHATSEEDVFRLLGLKYLPPEQRNA

SEQ ID NO: 11 - protein, 509 aa, Canis lupus
MDPLQMAHSGPRKKRPRQMGAPMVSPPHNIKFQDLVLYILEKKMGTTRRAFLMELARRKGFRVDNEFSDSITHIVAENNSGSDVLEWLQVQNIKASSQLELLDISWLIESMGAGKPVEMTGKHQLMRRDYTASPNPELQKTLPVAVKKISQYACQRRTTLNNYNNVFTDAFEVLAENYEFRENEVFSLTFMRAASVLKSLPFTIISMKDTEGIPCLGDQVKCIIEEIIEDGESSEVKAVLNDERYQSFKLFTSVFGVGLKTSEKWFRMGFRTLSKIKSDKSLKFTPMQKAGFLYYEDLVSCVTRAEAEAVGVLVKEAVGAFLPDAFVTMTGGFRRGKKMGHDVDFLITSPGSTDEDEEQLLPKVINLWERKGLLLYCDLVESTFEKLKLPSRKVDALDHFQKCFLILKLHHQRVDGGKCSQQEGKTWKAIRVDLVMCPYERRAFALLGWTGSRQFERDLRRYASHERKMILDNHALYDKTKKIFLKAESEEEIFAHLGLDYIEPWERNA

SEQ ID NO: 12 - protein, 506 aa, Gallus gallus
MERIRPPTVVSQRKRQKGMYSPKLSCGYEIKFNKLVIFIMQRKMGMTRRTFLMELARSKGFRVESELSDSVTHIVAENNSYPEVLDWLKGQAVGDSSRFEILDISWLTACMEMGRPVDLEKKYHLVEQAGQYPTLKTPESEVSSFTASKVSQYSCQRKTTLNNCNKKFTDAFEIMAENYEFKENEIFCLEFLRAASVLKSLPFPVTRMKDIQGLPCMGDRVRDVIEEIIEEGESSRAKDVLNDERYKSFKEFTSVFGVGVKTSEKWFRMGLRTVEEVKADKTLKLSKMQRAGFLYYEDLVSCVSKAEADAVSSIVKNTVCTFLPDALVTITGGFRRGKKIGHDIDFLITSPGQREDDELLHKGLLLYCDIIESTFVKEQIPSRHVDAMDHFQKCFAILKLYQPRVDNSSYNMSKKCDMAEVKDWKAIRVDLVITPFEQYAYALLGWTGSRQFGRDLRRYATHERKMMLDNHALYDKRKRVFLKAGSEEEIFAHLGLDYVEPWERNA

SEQ ID NO: 13 - protein, 509 aa, Homo sapiens
MDPPRASHLSPRKKRPRQTGALMASSPQDIKFQDLVVFILEKKMGTTRRAFLMELARRKGFRVENELSDSVTHIVAENNSGSDVLEWLQAQKVQVSSQPELLDVSWLIECIRAGKPVEMTGKHQLVVRRDYSDSTNPGPPKTPPIAVQKISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 14 - DNA, 14 bp, synthetic construct
  source 1..14: mol_type = other DNA
aaaaaaaaaagggg

SEQ ID NO: 15 - DNA, 10 bp, synthetic construct
  source 1..10: mol_type = other DNA
gtacgctagt

SEQ ID NO: 16 - DNA, 12 bp, synthetic construct
  source 1..12: mol_type = other DNA
  misc_binding >12: bound_moiety = At the 3' end of the sequence is a group which enables the sequnce to react covalently with a reaction group bound to a surface (e.g., NH2)
cctttttttttt